(19) United States
(12) Patent Application Publication
(10) Pub. No.: US 2021/0186930 A1
(43) Pub. Date: Jun. 24, 2021

(54) Methods of Treating Renal Disease Associated With Chronic Kidney Disease Such as Alport Syndrome

(71) Applicant: UNIVERSITY OF MIAMI, Miami, FL (US)

(72) Inventors: Alessia Fornoni, Miami, FL (US); Sandra Merscher, Miami, FL (US)

(21) Appl. No.: 17/057,247

(22) PCT Filed: May 14, 2019
(86) PCT No.: PCT/US19/32215

§ 371 Date: Nov. 20, 2020

Related U.S. Application Data
(60) Provisional application No. 62/675,537, filed on May 23, 2018.

(51) Int. Cl. A61K 31/397; A61P 13/12; A61K 31/403

(52) U.S. Cl. CPC A61K 31/397; A61K 31/403; A61P 13/12

(57) ABSTRACT

Disclosed herein is a method of treating renal disease in a subject suffering from Alport Syndrome or chronic kidney disease comprising administering ezetimibe (optionally in combination with ramipril) to the subject in an amount effective to treat the renal disease in the subject.

### FIELD OF THE INVENTION

The present disclosure relates to methods of treating renal disease in subjects suffering from chronic kidney disease such as Alport Syndrome.

### BACKGROUND

Alport Syndrome is an inherited disease caused by mutations in three collagen type IV genes, COL4A3, COL4A4, and COL4A5. The disease is characterized by progressive renal failure, hypertension, proteinuria, greatly increased risk of cardiovascular disease, and loss of hearing and vision. Current therapy for Alport Syndrome is aimed at ameliorating the symptoms and includes angiotensin-converting enzyme (ACE) inhibitors or angiotensin receptor blockers (ARB). Such treatments only slow the loss of kidney function or manage blood pressure and do not treat or prevent the other effects of the disease.

(3R,4S)-1-(4-Fluorophenyl)-3-[3(S)-3-(4-fluorophenyl)-3-hydroxypropyl)]-4-(4-hydroxyphenyl)-2-azetidinone (ezetimibe) is a cholesterol absorption inhibitor (U.S. Pat. No. 5,767,115). Preparation of ezetimibe is described in U.S. Pat. No. 5,767,115. One method of preparing ezetimibe comprises reacting (S)-4-phenyl-2-oxazolidinone with methyl-4-(chloroformyl)butyrate to obtain a compound of ester, which is condensed with 4-benzyloxy benzylidine (4-fluoro) aniline in the presence of titanium isopropoxide and titanium tetrachloride to give an amide compound. The intermediate is cyclised in the presence of tetrabutyl ammonium fluoride and bis trimethyl silyl acetamide to yield protected lactam, which undergoes hydrolysis to give a carboxylic acid, which is further reacted with p-fluoro phenyl magnesium bromide and zinc chloride in the presence of tetrakis (triphenyl phosphine) palladium to give an aromatic ketone. It is further reduced selectively in the presence of chiral catalyst to obtain an hydroxy compound, and undergoes debenzylation to yield the final product.

There remains a need for therapeutic options for Alport Syndrome and other Col4-related diseases or more broadly chronic kidney diseases to improve cardiac and kidney function, as well as hearing and vision.

### SUMMARY

In one aspect, described herein is a method of treating chronic kidney disease in a subject comprising administering ezetimibe to the subject in an amount effective to treat chronic kidney disease in the subject. In some embodiments, the method further comprises administering ramipril to the subject.

In another aspect, described herein is a method of treating renal disease in a subject suffering from Alport Syndrome comprising administering ezetimibe to the subject in an amount effective to treat renal disease in the subject. In some embodiments, the method further comprises administering ramipril to the subject.

In some embodiments, the ezetimibe reduces triglyceride content in a kidney of the subject. In some embodiments, the ezetimibe reduces kidney fibrosis in the subject. In some embodiments, the ezetimibe reduces mesangial expansion in the subject.

In another aspect, described herein is a method of improving renal function in a subject in need thereof comprising administering a combination of ezetimibe and ramipril to the subject in an amount effect to improve renal function. In some embodiments, the combination reduces estrerified cholesterol content in the subject.

In another aspect, described herein is a composition comprising ezetimibe and ramipril and a pharmaceutically acceptable carrier or diluent.

### BRIEF DESCRIPTION OF THE FIGURES

FIGS. 1A-H: Collagen treatment of human podocytes induces cytoskeletal rearrangement and intracellular lipid deposition. (A) F-actin staining was performed on collagen treated podocytes. Representative images are shown. Increased loss of stress fibers can be observed in collagen treated podocytes when compared to Ctrl podocytes. (B) Representative images of the collagen treated podocytes stained with BODIPY. (C) The number of lipid droplets per cell was counted using the OPERA high content screening system after labeling of intracellular lipids with BODIPY. Col I treated podocytes show an increased lipid droplet content (p<0.05). (D) Free fatty acid (FA) uptake measured using fluorescent labeled free fatty acid is increased in collagen I treated podocytes (***p<0.001). (E) Cellular triglyceride content is increased in podocytes after Col I treatment (n=1, duplicate, p<0.01). (F,G,H) An increased total cholesterol content (F) and esterified cholesterol content (G) can be observed in Col I treated podocytes, while the cholesterol ester to total cholesterol ratio seems mildly decreased (H).

FIGS. 2A-F: DDR1 is activated by Collagen I and DDR1 activation leads to increased FFA uptake via CD36. (A) Treatment of normal human podocytes with Col I leads to DDR1 activation (pDDR1). (B) Treatment of human podocytes with Collagen I but not Col IV leads to activation of DDR1 at the plasma membrane. (C) Protein expression of CD36 is increased whereas FASN and CPT1A remain unchanged in HEK293 cells transfected with GFP-tagged DDR1 DA (dominant active) compared to DDR1 WT (wildtype) and DDR1 DN (dominant negative). (D) Uptake of the free fatty acids is increased in HEK293 cells transfected with DDR1 DA and decreased in DDR1 DN transfected HEK cells, (*p<0.05). (E) Uptake of the free fatty acid is decreased in CD36 KD cells when compared to control cells (*p<0.05). (F) Co-immunoprecipitation analysis showing that GFP-DDR1 WT, DA, and DN precipitate with FLAG-tagged CD36 from cotransfected HEK293 cells. All GFP-tagged DDR1 proteins interact with CD36. No binding was found with GFP N1 (control).

FIGS. 3A-D: ezetimibe protects Col4a3 knockout mice from renal failure. (A) ezetimibe treatment of Col4a3 knockout mice results in a significant reduction in the albumin/creatinine ratio compared to untreated Col4a3 knockout mice. **p<0.01, **p<0.001. (B) Serum creatinine levels are significantly increased in Col4a3 knockout mice compared to controls. ezetimibe treatment significantly reduces serum creatinine levels in Col4a3 knockout mice. *p<0.05, t-test, **p<0.01. (C) Serum BUN levels are significantly increased in Col4a3 knockout mice compared to controls whereas ezetimibe treatment prevents increases in serum BUN levels in Col4a3 knockout mice. *p<0.05, **p<0.01, t test. (D) Body weight is significantly reduced in Col4a3 knockout mice compared to controls. ezetimibe prevents body weight loss in Col4a3 knockout mice *p<0.05, ****p<0.0001.

FIGS. 4A-J: ezetimibe protects Col4a3 knockout mice from fibrosis and changes in plasma and kidney lipids. (A) Representative image of PicroSirius Red staining demonstrating increased fibrosis in Col4a3 knockout mice compared to ezetimibe (Cpd) treated Col4a3 knockout mice. (B) Bar graph analysis showing that ezetimibe treatment of Col4a3 knockout mice results in a significant reduction of fibrosis. *p<0.05, ****p<0.0001. (C) Representative image of Oil Red 0 staining demonstrating increased lipid droplet accumulation in Col4a3 knockout mice compared to ezetimibe (Cpd) treated Col4a3 knockout mice. (D) Bar graph analysis showing that ezetimibe treatment of Col4a3 knockout mice results in a significant reduction of glomerular lipid droplets. **p<0.01. (E, F) Total serum cholesterol (E) and triglyceride (F) levels are increased in Col4a3 knockout mice but significantly decreased in ezetimibe treated Col4a3 knockout mice. *p<0.05, ****p<0.0001 (G) No change in total cholesterol levels is observed in kidney cortexes of Col4a3 knockout mice treated with ezetimibe or untreated. (H) ezetimibe treatment significantly reduces triglyceride content in kidney cortexes of Col4a3 knockout mice. *p<0.05. (I) The ratio of esterified to total cholesterol is significantly increased in kidney cortexes of ezetimibe treated and untreated Col4a3 knockout mice when compared to controls. *p<0.05. (J) CD36 expression is significantly increased in Col4a3 knockout mice and in ezetimibe treated Col4a3 knockout mice **p<0.01.

FIGS. 5A-5C show that ezetimibe and ramipril improve renal function in an in vivo model of Alport syndrome but only ramipril or the combination of ezetimibe and ramipril reduces the esterified cholesterol content. (FIG. 5A) Oral administration of ezetimibe, ramipril or of both drugs combined to Col4a3−/− mice results in a significant reduction in the albumin/creatinine ratio (ACR) when compared to untreated Col4a3−/− mice. The combination of ezetimibe and ramipril does not have a superior effect on the reduction of the albumin/creatinine ratio when compared to ezetimibe or ramipril alone. *p<0.05, **p<0.01; ***p<0.001, ****p<0.0001, t-test, *p<0.05, one-way ANOVA. (FIG. 5B) Oral administration of ezetimibe, ramipril or of both drugs combined to Col4a3−/− mice results in a significant reduction in the serum creatinine levels when compared to untreated Col4a3−/− mice. The combination of ezetimibe and ramipril does not have a superior effect on the reduction of the serum creatinine when compared to ramipril alone but ramipril alone has a superior effect on reducing serum creatinine levels compared to ezetimibe alone. *p<0.05, **p<0.01; ***p<0.001, t-test, *p<0.05, one-way ANOVA. (FIG. 5C) Oral administration of ezetimibe, ramipril or of both drugs combined to Col4a3−/− mice results in a significant reduction in the serum BUN levels when compared to untreated Col4a3−/− mice. The combination of ezetimibe and ramipril does not have a superior effect on the reduction of the BUN levels when compared to ramipril alone but ramipril alone has a superior effect on reducing BUN levels compared to ezetimibe alone. *p<0.05, **p<0.01; ***p<0.001, t-test, *p<0.05, one-way ANOVA.

FIGS. 6A-6D show that ezetimibe, ramipril and ezetimibe+ramipril improve mesangial expansion and renal fibrosis in an in vivo model of Alport syndrome. Oral administration of ezetimibe, ramipril and ezetimibe+ramipril to Col4a3−/− mice resulted in a significant reduction in mesangial expansion and renal fibrosis when compared to untreated Col4a3−/− mice. (FIG. 6A) Representative image of PAS staining. (FIG. 6B) Representative image of Picrossirius Red staining. (FIG. 6C, FIG. 6D) Bar graph quantification of the mesangial expansion score (FIG. 6C) and of renal fibrosis (FIG. 6D). *p<0.05; ; **p<0.01, ; ***p<0.001****p<0.0001, t-test. *p<0.05, one-way ANOVA.

FIGS. 7A and 7B show that ramipril affects renal lipid content in an in vivo model of Alport syndrome. (FIG. 7A) Esterified cholesterol (CE) content in kidney cortexes is significantly decreased in ramipril and ramipril+ezetimibe treated Col4a3−/− mice when compared to untreated Col4a3−/− mice, while ezetimibe treatment only has no effect on the total cholesterol content. (FIG. 7B) Triglyceride (TG) content in kidney cortexes is significantly decreased in ezetimibe treated Col4a3−/− mice when compared to untreated Col4a3−/− mice while ramipril and ramipril+ezetimibe treatment has no effect on the Triglyceride content. *p<0.05; **p<0.01, t-test. *p<0.05, one-way ANOVA. EZ=ezetimibe, Ramp=ramipril.

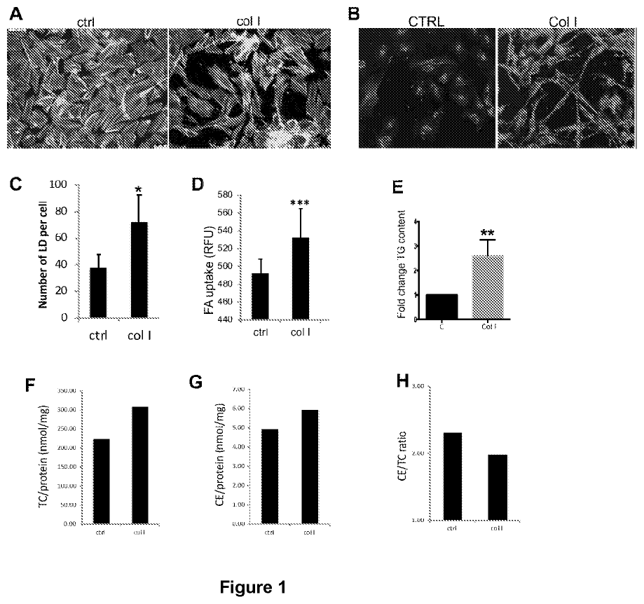

### DETAILED DESCRIPTION

The present disclosure provides methods of treating renal disease in a subject suffering from Alport Syndrome in need thereof comprising administering a therapeutically effective amount of (3R,4S)-1-(4-Fluorophenyl)-3-[3(S)-3-(4-fluorophenyl)-3-hydroxypropyl)]-4-(4-hydroxyphenyl)-2-azetidinone, referred to herein as ezetimibe, optionally in combination with (2S,3aS,6aS)-1[(S)—N—[(S)-1-Carboxy-3-phenylpropyl]alanyl]octahydrocyclop-enta[b]pyrrole-2-carboxylic acid, 1-ethyl ester, referred to herein as ramipril.

Ezetimibe is sold under the brand name Zetia®, which is marketed by Merck/Schering-Plough Pharmaceuticals. Zetia® is available as a tablet for oral administration containing 10 mg of ezetimibe. The inactive ingredients of Zetia® are reported to be croscarmellose sodium, lactose monohydrate, magnesium stearate, microcrystalline cellulose, povidone, and sodium lauryl sulfate. The recommended dose is 10 mg once daily, administered with or without food, according to the Zetia® label.

Ezetimibe, a white crystalline powder disclosed in, e.g., European Patent No. 0 720 599 and U.S. Pat. No. 5,631,365), is reported to be freely to very soluble in ethanol, methanol, and acetone, and practically insoluble in water. Further it is reported to have a melting point of about 163° C. and to be stable at ambient temperature. The mechanism of action of ezetimibe on absorption and resorption inhibition of cholesterol involves increased excretions of cholesterol and its intestinally generated metabolites with the feces. This effect results in lowered body cholesterol levels, increased cholesterol synthesis, and decreased triglyceride synthesis. The increased cholesterol synthesis initially provides for the maintenance of cholesterol levels in the circulation, levels that eventually decline as the inhibition of cholesterol absorption and resorption continues. The overall effect of drug action is the lowering of cholesterol levels in the circulation and tissues of the body.

Rampiril is an angiotensin-converting-enzyme (ACE) inhibitor used in the treatment of cardiovascular disease, especially hypertension and nephropathia, and congestive heart failure. In hypertensive patients, ramipril is known to cause a reduction in peripheral arterial resistance, and thus, a reduction in blood pressure without a compensatory rise in heart rate. Ramipril has also been shown to reduce mortality in patients with clinical signs of congestive heart failure after surviving an acute myocardial infarction. Ramipril has been suggested to have an added advantage over many other ACE inhibitors due to its pronounced inhibition of the ACE enzymes in tissues resulting in organ protective effects, e.g., in the heart, kidney, and blood vessels.

Ramipril, a 2-aza-bicyclo [3.3.0]-octane-3-carboxylic acid derivative, is a white, crystalline particular substance or powder that is soluble in polar organic solvents and buffered aqueous solutions. The ramipril crystalline particles are columnar (or needle like) in shape. The ramipril crystalline particles melt between about 105° C. and about 112° C. ramipril and processes for making and using ramipril are described in U.S. Pat. Nos. 4,587,258, 5,061,722 and 5,403,856, all of which are incorporated herein by reference in their entirety. The preparation of ramipril has also been described in EP 0 079 022 A2, EP 0 317 878 A1 and DE 44 20 102 A, which are incorporated herein by reference in their entirety.

In certain embodiments, the subject has been diagnosed as having Alport Syndrome prior to administration of ezetimibe and/or ramipril. Diagnosis of Alport Syndrome may be achieved through evaluation of parameters including, without limitation, a subject's family history, clinical features (including without limitation proteinuria, albuminuria, hematuria, impaired GFR, deafness and/or ocular changes) and results of tissue biopsies. Kidney biopsies may be tested for the presence or absence of the type IV collagen alpha-3, alpha-4, and alpha-5 chains. Additionally, structural changes in the glomerulus can be detected by electron microscopy of kidney biopsy material. A skin biopsy may be tested for the presence of the type IV collagen alpha-5 chain, which is normally present in skin and almost always absent from male subjects with the X-linked form of Alport Syndrome. Diagnosis of Alport Syndrome may also include screening for mutations in one or more of the Col4a3, Col4a4, or Col4a5 genes.

The term “renal disorder,” “renal disease” or “kidney disease” as used herein means any alteration in normal physiology and function of the kidney. This term includes but is not limited to diseases and conditions such as kidney transplant; nephropathy; primary glomerulopathies (focal segmental glomerulosclerosis), Minimal Change disease, Membranous GN, IgA Nephropathy, chronic kidney disease (CKD); Glomerulonephritis; inherited diseases such as polycystic kidney disease; Acute and chronic interstitial nephritis, Mesoamerican Nephropathy, nephromegaly (extreme hypertrophy of one or both kidneys); nephrotic syndrome; Nephritic syndrome, end stage renal disease (ESRD); acute and chronic renal failure; interstitial disease; nephritis; sclerosis, an induration or hardening of tissues and/or vessels resulting from causes that include, for example, inflammation due to disease or injury; renal fibrosis and scarring; renal-associated proliferative disorders; and other primary or secondary nephrogenic conditions.

The term “chronic kidney disease (CKD)” as used herein is defined as abnormalities of kidney structure or function, present for more than three months, with implications for health. CKD has been classified according to the National Kidney Foundation developed criteria into 5 stages, where stage 1 is kidney damage with normal eGFR (mL/min/1.73 m²) of 90 or above; stage 2 is kidney damage with a mild decrease in GFR (GFR 60-89 mL/min/1.73 m²); stage 3 is a moderate decrease in GFR (GFR 30-59 mL/min/1.73 m²); stage 4 is a severe decrease in GFR (GFR 15-29 mL/min/1.73 m²); and stage 5 is kidney failure (GFR<15 mL/min/1.73 m² or dialysis). Stage 3 has been subdivided into stage 3A, which is a mild to moderate decrease in GFR (GFR 45-59), and stage 3B, which is a moderate to severe decrease in GFR (GFR 30-44).

Renal disorders or kidney diseases may also be generally defined as a “nephropathy” or “nephropathies.” The terms “nephropathy” or “nephropathies” encompass all clinical-pathological changes in the kidney which may result in kidney fibrosis and/or glomerular diseases (e.g., glomerulosclerosis or glomerulonephritis) and/or chronic renal insufficiency, and can cause end stage renal disease and/or renal failure. Some aspects of the present disclosure relate to compositions and their uses for the prevention and/or treatment of hypertensive nephropathy, diabetic nephropathy, and other types of nephropathy such as analgesic nephropathy, immune-mediated glomerulopathies (e.g., IgA nephropathy or Berger's disease, lupus nephritis), ischemic nephropathy, HIV-associated nephropathy, membranous nephropathy, glomerulonephritis, glomerulosclerosis, radiocontrast media-induced nephropathy, toxic nephropathy, analgesic-induced nephrotoxicity, cisplatin nephropathy, transplant nephropathy, and other forms of glomerular abnormality or injury, or glomerular capillary injury (tubular fibrosis). In some embodiments, the terms “nephropathy” or “nephropathies” refer specifically to a disorder or disease where there is either the presence of proteins (i.e., proteinuria) in the urine of a subject and/or the presence of renal insufficiency.

In some embodiments, the subject is suffering from albuminuria or proteinuria. Exemplary disorders associated with albuminuria include, but are not limited to, chronic kidney disease, proliferative glomerulonephritis (e.g., immunoglobulin A nephropathy, membranoproliferative glomerulonephritis, mesangial proliferative glomerulonephritis, anti-GBM disease, renal vasculitis, lupus nephritis, cryoglobulinemia-associated glomerulonephritis, bacterial endocarditis, Henoch-Schonlein purpura, postinfectious glomerulonephritis, or hepatitis C), and nonproliferative glomerulonephritis (e.g., membranous glomerulonephritis, minimal-change disease, primary focal segmental glomerulosclerosis (FSGS), fibrillary glomerulonephritis, immunotactoid glomerulonephritis, amyloidosis, hypertensive nephrosclerosis, light-chain disease from multiple myeloma and secondary focal glomerulosclerosis).

In any of the embodiments provided herein, a subject may be subjected to certain tests to evaluate kidney function. Such tests include, without limitation, measurement of blood urea nitrogen in the subject; measuring creatinine in the blood of the subject; measuring creatinine clearance in the blood of the subject; measuring proteinuria in the subject; measuring albumin:creatinine ratio in the subject; measuring glomerular filtration rate in the subject; and measuring urinary output in the subject.

In any of the embodiments provided herein, proteins present in the urine or blood may be used to evaluate kidney function. Such tests of kidney function include, but are not limited to, measuring albumin/creatinine urinary ratios, total protein/creatinine urinary ratios, albumin or total proteins in timed urinary collections, N-acetyl-β-D-glucosaminidase (NAG) protein in the urine of the subject; measuring neutrophil gelatinase-associated lipocalin (NGAL) protein in the urine of the subject; measuring kidney injury molecule-1 (KIM-1) protein in the urine of the subject; measuring interleukin-18 (IL-18) protein in the urine of the subject; measuring connective tissue growth factor (CTGF) levels in the urine of the subject; measuring monocyte chemoattractant protein 1 (MCP1) levels in the urine of the subject; measuring collagen IV (Col IV) fragments in the urine of the subject; measuring collagen III (Col III) fragment levels in the urine of the subject; measuring cystatin C protein in the blood of a subject; measuring β-trace protein (BTP) in the blood of a subject; and measuring 2-microglobulin (B2M) in the blood of a subject. In any of the embodiments provided herein, markers of podocyte injury can be measuring in the urine. Such proteins include nephrin and podocin. The proteins may be quantitated, for example, by enzyme-linked immunosorbent assay (ELISA), or radioimmunoassay (RIA) using commercially available kits.

Timing of Administration and Dosage

In some embodiments, one or more administrations of ezetimibe (optionally in combination with rampiril) described herein are carried out over a therapeutic period of, for example, about 1 week to about 18 months (e.g., about 1 month to about 12 months, about 1 month to about 9 months or about 1 month to about 6 months or about 1 month to about 3 months). In some embodiments, a subject is administered one or more doses of ezetimibe described herein over a therapeutic period of, for example, about 1 month to about 12 months (52 weeks) (e.g., about 2 months, about 3 months, about 4 months, about 5 months, about 6 months, about 7 months, about 8 months, about 9 months, about 10 months, or about 11 months).

In addition, it may be advantageous to administer multiple doses of the ezetimibe or separate the administration of doses in time, depending on the therapeutic regimen selected for a particular human subject. In some embodiments, the ezetimibe is administered periodically over a time period of one year (12 months, 52 weeks) or less (e.g., 9 months or less, 6 months or less, or 3 months or less). In this regard, ezetimibe is optionally administered to the human once every about 3 days, or about 7 days, or about 2 weeks, or about 3 weeks, or about 4 weeks, or about 5 weeks, or about 6 weeks, or about 7 weeks, or about 8 weeks, or about 9 weeks, or about 10 weeks, or about 11 weeks, or about 12 weeks, or about 13 weeks, or about 14 weeks, or about 15 weeks, or about 16 weeks, or about 17 weeks, or about 18 weeks, or about 19 weeks, or about 20 weeks, or about 21 weeks, or about 22 weeks, or about 23 weeks, or about 6 months, or about 12 months.

In some embodiments, a dose of ezetimibe comprises between about 1 to about 500 milligrams (e.g., between about 1 to about 400 milligrams or about 3 to about 300 milligrams) of ezetimibe per kilogram of body weight (mg/kg). For example, the dose of ezetimibe may comprise at least about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, or about 49 mg/kg, or about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 125 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 450 mg/kg, or about 500 mg/kg. Ranges between any and all of these endpoints are also contemplated, e.g., about 1 mg/kg to about 100 mg/kg, about 3 mg/kg to about 300 mg/kg, about 3 mg/kg to about 100 mg/kg, about 5 mg/kg to about 50 mg/kg, about 3 mg/kg to about 75 mg/kg, about 1 mg/kg to about 50 mg/kg, about 100 mg/kg to about 300 mg/kg, about 50 mg/kg to about 200 mg/kg, or about 200 mg/kg to about 300 mg/kg.

In some embodiments, one or more administrations of ramipril described herein are carried out over a therapeutic period of, for example, about 1 week to about 18 months (e.g., about 1 month to about 12 months, about 1 month to about 9 months or about 1 month to about 6 months or about 1 month to about 3 months). In some embodiments, a subject is administered one or more doses of ramipril described herein over a therapeutic period of, for example, about 1 month to about 12 months (52 weeks) (e.g., about 2 months, about 3 months, about 4 months, about 5 months, about 6 months, about 7 months, about 8 months, about 9 months, about 10 months, or about 11 months).

In addition, it may be advantageous to administer multiple doses of the ramipril or separate the administration of doses in time, depending on the therapeutic regimen selected for a particular human subject. In some embodiments, the ramipril is administered periodically over a time period of one year (12 months, 52 weeks) or less (e.g., 9 months or less, 6 months or less, or 3 months or less). In this regard, ramipril is optionally administered to the human once every about 3 days, or about 7 days, or about 2 weeks, or about 3 weeks, or about 4 weeks, or about 5 weeks, or about 6 weeks, or about 7 weeks, or about 8 weeks, or about 9 weeks, or about 10 weeks, or about 11 weeks, or about 12 weeks, or about 13 weeks, or about 14 weeks, or about 15 weeks, or about 16 weeks, or about 17 weeks, or about 18 weeks, or about 19 weeks, or about 20 weeks, or about 21 weeks, or about 22 weeks, or about 23 weeks, or about 6 months, or about 12 months.

In some embodiments, a dose of ramipril comprises between about 1 to about 500 milligrams (e.g., between about 1 to about 400 milligrams or about 3 to about 300 milligrams) of ramipril per kilogram of body weight (mg/kg). For example, the dose of ramipril may comprise at least about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, or about 49 mg/kg, or about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 125 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 450 mg/kg, or about 500 mg/kg. Ranges between any and all of these endpoints are also contemplated, e.g., about 1 mg/kg to about 100 mg/kg, about 3 mg/kg to about 300 mg/kg, about 3 mg/kg to about 100 mg/kg, about 5 mg/kg to about 50 mg/kg, about 3 mg/kg to about 75 mg/kg, about 1 mg/kg to about 50 mg/kg, about 100 mg/kg to about 300 mg/kg, about 50 mg/kg to about 200 mg/kg, or about 200 mg/kg to about 300 mg/kg.

In some embodiments, the ezetimibe and ramipril are administered concurrently (e.g., either in the same composition, or administered in separate compositions but at the same time). In some embodiments, the ezetimibe and ramipril are administered sequentially (e.g., within 1 hour, 2 hours, 3 hours, 4 hours, 5 hours, 6 hours, 12 hours, 24 hours, 36 hours or 48 hours of each other).

Pharmaceutical Compositions

In some embodiments, ezetimibe and/or ramipril is formulated with a pharmaceutically effective diluents, carrier, solubilizer, emulsifier, preservative, and/or adjuvant. Pharmaceutical compositions include, but are not limited to, liquid, frozen, and lyophilized compositions.

Preferably, formulation materials are nontoxic to recipients at the dosages and concentrations employed. In specific embodiments, pharmaceutical compositions comprising a therapeutically effective amount of ezetimibe and/or ramipril are provided.

In some embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

Combination Therapies

In some embodiments, the methods described herein comprise administering another agent for preventing or treating a renal disorder such as nephropathy, or an associated disorder or complication. Examples of such known compounds include but are not limited to: ACE inhibitor drugs (e.g., ramipril, captopril (Capoten®), enalapril (Innovace®), fosinopril (Staril®), lisinopril (Zestril®), perindopril (Coversyl®), quinapril (Accupro®), trandanalopril (Gopten®), lotensin, moexipril, ramipril); RAS blockers; angiotensin receptor blockers (ARBs) (e.g., Olmesartan, Irbesartan, Losartan, Valsartan, candesartan, eprosartan, telmisartan, etc); protein kinase C (PKC) inhibitors (e.g., ruboxistaurin); inhibitors of AGE-dependent pathways (e.g., aminoguanidine, ALT-946, pyrodoxamine (pyrododorin), OPB-9295, alagebrium); anti-inflammatory agents (e.g., clyclooxigenase-2 inhibitors, mycophenolate mophetil, mizoribine, pentoxifylline), GAGs (e.g., sulodexide (U.S. Pat. No. 5,496,807)); pyridoxamine (U.S. Pat. No. 7,030,146); endothelin antagonists (e.g., SPP 301), COX-2 inhibitors, PPAR-γ antagonists and other compounds like amifostine (used for cisplatin nephropathy), captopril (used for diabetic nephropathy), cyclophosphamide (used for idiopathic membranous nephropathy), sodium thiosulfate (used for cisplatin nephropathy), tranilast, etc. (Williams and Tuttle (2005), Advances in Chronic Kidney Disease, 12 (2):212-222; Giunti et al. (2006), Minerva Medica, 97:241-62).

Additionally, the methods described herein may also include co-administration of at least one other therapeutic agent for the treatment of another disease directly or indirectly related to renal disorder complications, including but not limited to: dyslipidemia, hypertension, obesity, neuropathy, inflammation, and/or retinopathy. Such additional therapeutic agents include, but are not limited to, corticosteroids; immunosuppressive medications; antibiotics; antihypertensive and diuretic medications (such as thiazide diuretics and ACE-inhibitors or β-adrenergic antagonists); lipid lowering agents such as bile sequestrant resins, cholestyramine, colestipol, nicotinic acid, and more particularly drugs and medications used to reduce cholesterol and triglycerides (e.g., fibrates (e.g., Gemfibrozil®) and HMG-CoA inhibitors such as Lovastatin®, Atorvastatin®, Fluvastatin®, Lescol®, Lipitor®, Mevacor®, Pravachol®, Pravastatin®, Simvastatin®, Zocor®, Cerivastatin®, etc); nicotinic acid; and Vitamin D.

In some embodiments, the ezetimibe is not administered in combination of a ceramide.

Additional examples of agents that can be co-administered with ezetimibe (and/or ramipril) described herein include immunomodulating agents or immunouppressants (such as those that are used by subjects who have received a kidney transplant (e.g., when they have developed a nephropathy), anti-obesity agents, and appetite reducers (including, but not limited to, Xenical™ (Roche), Meridia™ (Abbott), Acomplia™ (Sanofi-Aventis), and sympathomimetic phentermine), agents that are used to treat hyperkalemia and/or to reduce the risk of ventricular fibrillation caused by hyperkalemia (e.g., calcium gluconate, insulin, sodium bicarbonate, β₂-selective catacholamine such as salbutamol (albuterol, Ventolin®), and polystyrene sulfonate (Calcium Resonium, Kayexalate)) and patiromer (Veltassa®).

As used herein, the term “concomitant” or “concomitantly” as in the phrases “concomitant therapeutic treatment” or “concomitantly with” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g., a human). Preferably the first agent is ezetimibe and/or ramipril. The second agent may be selected from the other therapeutics described herein.

### EXAMPLES

Materials and Methods:

Human podocyte culture: A human podocyte cell line transfected with a thermosensitive SV40-T construct was cultured as previously described (Saleem, M. A., et al. A conditionally immortalized human podocyte cell line demonstrating nephrin and podocin expression. J Am Soc Nephrol 13, 630-638, 2002). Differentiated human podocytes were serum starved for 16 hours followed by 18 hours treatment with 50 μg/mL collagen I (Corning), collagen II (Discoverx), and collagen IV (Sigma Aldrich)

Lipid content determination: Following collagen treatment, podocyte lipid content was assessed after fixation with PFA and staining with Bodipy 493/503 and Cell Mask Blue (Invitrogen). The Opera High Content Screening system and Acapella Image Analysis software was used to determine the number of Bodipy 493/503 positive lipid droplets per cell. Triglyceride content was assessed using the Triglyceride Quantification Kit (Cell Biolabs) following manufacturer instructions and normalizing to cell protein content using the BCA method.

Free fatty acid uptake was assessed using the fluorometric free fatty acid uptake Kit (Abcam) in Ctrl and Col I treated podocytes after 1 hour serum starvation. The uptake of FFA was analyzed using a fluorimeter (SpectraMax5, Molecular Probes).

Co-Immunoprecipitation (Co-IP) experiments of exogenous and endogenous proteins were performed: The DDR1-CD36 protein-protein interaction was assessed by Co-IP analysis. For exogenous co-IP experiments, HEK 293 cells were co-transfected with FLAG-tagged CD36 and GFP-tagged DDR1 WT, DA, and DN plasmids. Immunoprecipitates of transfected HEK cell lysates were prepared using FLAG beads (Sigma-Aldrich) and the interaction between the proteins was confirmed by Western blot analysis using GFP antibody (Clonetech).

Isolation of plasma membranes: Preparation of membrane pellets was performed by ultracentrifugation of cell pellets suspended in homogenization media (15 mM KCl, 1.5 mM MgCl2, 10 mM HEPES, 1 mM DTT) supplemented with protease inhibitors. Effective separation was verified by WB for Na-K ATPase.

Determination of cholesterol content in kidney cortex: Tissue from kidney cortexes was homogenized in hypotonic buffer (10 mM HEPES pH7.0, 15 mM KCl, 1 mM MgCl₂, 10 mM phosphatase inhibitors). Lipids from 100 μl of the homogenate were extracted with 1 ml of hexane:isopropanol (3:2, v/v), dried and reconstituted in 100 μl of isopropanol-NP40 (9:1, v/v). Tissue fractions not soluble in the organic solvent, were further dried, reconstituted in 8 M urea, and assayed for protein concentration using Pierce BCA protein assay kit (ThermoFisher Scientific, MA, USA). Total cholesterol from lipid extracts was determined by the enzymatic method as previously described (Robinet, P., Wang, Z., Hazen, S. L. & Smith, J. D. A simple and sensitive enzymatic method for cholesterol quantification in macrophages and foam cells. Journal of lipid research 51, 3364-3369, 2010). Briefly, endogenous peroxides present were eliminated by incubating samples and standards with catalase (Sigma-Aldrich, MO, USA), 45 U/ml final concentration, for 15 min at 37° C. Then, 50 μl of samples and standards were mixed with 50 μl of reagent A (2 U/ml cholesterol oxidase (Sigma-Aldrich, MO, USA), 0.6 U/ml cholesterol esterase (MP Biomedicals), 2 U/ml HRP (Sigma), 100 μM Amplex Red (ThermoFisher Scientific, MA, USA) in black 96-well plates. The plates were incubated at 37° C. for 30 min and fluorescence was read at 530 nm (Ex) and 580 nm (Em) in a plate reader (SpectraMax M5, Molecular Devices, CA, USA). Cholesteryl esters (CE) quantification was based on the method described from Toshimi M, et al. Briefly, 25 μl of samples was mixed with 150 μl of free cholesterol decomposition reagent (45 U/ml catalase, 1 U/ml cholesterol oxidase) and incubated 2-4 h at 37° C., and then the final volume was brought up to 250 μl prior to determining free and total cholesterol remaining. Total cholesterol remaining was determined by mixing 50 μl of end product reaction with 50 μl of reagent A, as described above. Free cholesterol remaining was determined similarly, but without including cholesterol esterase in the reagent. CE was calculated by the formula CE=(TC−FC)×DF, where TC is the total cholesterol remaining, FC the free cholesterol remaining and DF the dilution factor, in this case, 10.

Lipid content: Oil Red 0 staining was performed on frozen tissue sections and triglyceride content was determined on snap frozen kidney tissue as described above.

Mouse models: Mice in which exon 5 of a3 chain of collagen type IV is deleted (Col4a3 KO) were obtained from the Jackson Laboratory (#002908, 129-Col4a3tm1Dec/J). Col4a3 heterozygous littermates were bred to generate Col4a3 KO mice. Four groups of mice with n=3-10 per group were studied. The four groups consisted of: 1) wildtype controls+vehicle, 2) wildtype controls+ezetimibe, 3) Col4a3 KO+vehicle, 4) Col4a3 KO+ezetimibe. ezetimibe was administered by oral gavage daily starting at 4 weeks of age at a concentration of 5 mg/kg body weight. Body weight and albuminuria were monitored weekly starting at the age of 4 weeks to 16 weeks. Mice were sacrificed at 8 weeks of age when progression to end-stage renal failure had occurred.

Albumin/creatinine ratio: All albuminuria values are expressed as μg albumin/mg creatinine. Albumin/creatinine determined by ELISA (Bethyl Laboratories) for albumin detection and a biochemical assay based on the Jaffe method for creatinine detection (Stanbio) as previously described.

Serology: Immediately prior to sacrifice, a blood sample was collected for the determination of BUN and lipid panel analysis in the comparative laboratory core facility of the University of Miami. Serum creatinine was determined by tandem mass spectrometry (UAB-UCSD O'Brien Core Center, University of Alabama).

Mouse sacrifice surgery: Mice were perfused through the left ventricle with phosphate buffered saline at 8 weeks of age. The right kidney was removed, one pole excised and embedded in OCT for Stochastic Optical Reconstruction Microscopy (STORM), the remainder of the kidney was used for cholesterol content determination and mRNA extraction from cortexes. The two poles of the left kidney were excised and fixed in 4% PFA, one pole was used for paraffin-embedding followed by histological analysis, other pole was post-fixed for 12h in 4% Glutaraldehyde solution in PBS for electron microscopy.

Immunofluorescence staining: After blocking, frozen tissue sections were incubated with rabbit anti DDR1 (1:50, Santacruz) and mouse monoclonal anti synaptopodin overnight at 40° C. After exposure to the secondary antibody in the dark for 90 min at RT, Goat anti rabbit Alexa Fluor 488 (Invitrogen, 1:400) and Goat anti mouse Alexa Fluor 555 (Invitrogen, 1:500) were utilized prior to addition of DAPI enriched mounting media.

Assessment of mesangial expansion: Periodic acid-Schiff (PAS) staining of 4 μm-thick tissue sections was performed using a standard protocol. Twenty glomeruli per section were analyzed for mesangial expansion by semi quantitative analysis (scale 0-4) performed by two blinded independent investigators.

Picrosirius red staining: Paraffin-embedded sections (4 μm thick) were deparaffinized with xylene and a graded alcohol series. Sections were rinsed for 5 min in distilled water, stained for 1 hour with picrosirius red in saturated aqueous picric acid, examined under a light microscope and photomicrographs were taken. Histological images were visualized using a light microscope (Olympus BX 41, Tokyo, Japan) at 40× magnification and analyzed using Image J software.

Statistical Analysis: All in vitro experiments were performed in triplicates and 3 biological replicates were performed, in all in vivo experiments, 3-10 mice per group were analyzed. Statistical Analysis was implemented using Graph Pad Prism Software. Analysis of Variance (ANOVA) followed by Bonferroni's posttest or Student's t-test was used to analyze results.

The data generated herein demonstrates that Col1-induced or genetic activation of DDR1 causes cellular toxicity. In particular, the data here demonstrates that the uptake of free fatty acids (FFAs) is increased in Col I treated podocytes and requires the activation of DDR1 which will ultimately lead to intracellular lipid accumulation due to increased fatty acid (FA) uptake. This phenomenon was also reflected in Col4a3 KO mice, where increased DDR1 activity in kidney cortexes correlated with blood uring nitrogen (BUN) and is associated with increased lipid deposition and increased expression of scavenger receptor B, cluster of differentiation 36 (CD36), a protein involved in FA uptake, cholesterol absorption and activation of inflammatory pathways.

### Example 1—Ezetimibe, Rampiril and the Ezetimibe/Rampiril Combination Improve Renal Function Col4a3 Mice

The following experiment was performed to investigate which treatment, ezetimibe (EZ), ramipril (Ramp) or the combination of both drugs (EZ+Ramp), would best preserve renal function in Col4a3 knockout mice, all drugs were administered to 4-week-old Col4a3−/− mice for 4 weeks as follows: ezetimibe (Ez) was orally administered at a concentration of 5 mg/kg BW, ramipril was added to the drinking water at a concentration that would lead to a daily ramipril uptake of 10 mg/kg BW. All mice were sacrificed at 8 weeks of age. The following four groups of mice were analyzed: Col4a3^{+/+}, Col4a3^{-/-}+Ez, Col4a3^{-/-}+Ramp and Col4a3^{-/-}+Ez+Ramp.

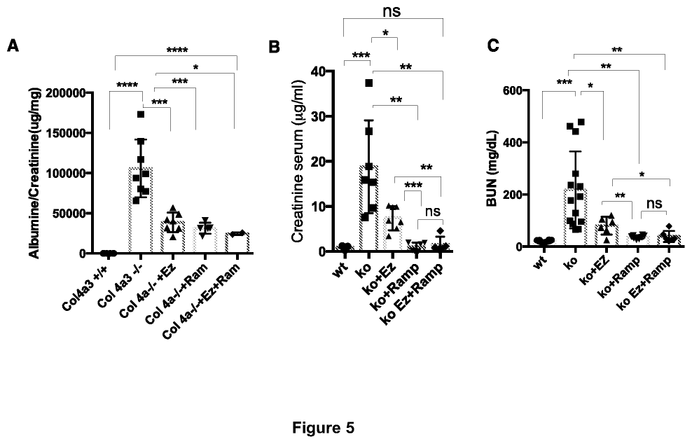

A significant decrease in the albumin/creatinine ratio (ACR) in Col4a3^{-/-} mice treated with ezetimibe, ramipril or of both drugs combined when compared to untreated Col4a3−/− mice. While each drug alone was very effective in reducing the ACR, the combination of ezetimibe and ramipril did not have a superior effect (FIG. 5A). Oral administration of ezetimibe, ramipril or of both drugs combined to Col4a3−/− mice also resulted in a significant reduction in the serum creatinine levels when compared to untreated Col4a3−/− mice. While the combination of ezetimibe and ramipril did not have a superior effect on the reduction of serum creatinine levels when compared to treatment with ramipril alone, a superior effect on reducing serum creatinine levels compared to ezetimibe alone was observed (FIG. 5B). A similar effect on BUN levels was observed where oral administration of ezetimibe, ramipril or of both drugs combined to Col4a3^{-/-} mice resulted in a significant reduction in the serum BUN levels when compared to untreated Col4a3^{-/-} mice while the combination of ezetimibe and ramipril did not have a superior effect on the reduction of the BUN levels when compared to ramipril alone. With regard to BUN levels, ramipril alone had a superior effect on reducing BUN levels when compared to ezetimibe alone (FIG. 5C).

### Example 3—Ezetimibe, Ramipril and the Ezetimibe/Ramipril Combination Improve Mesangial Expansion and Renal Fibrosis in Col4a3 Knockout Mice

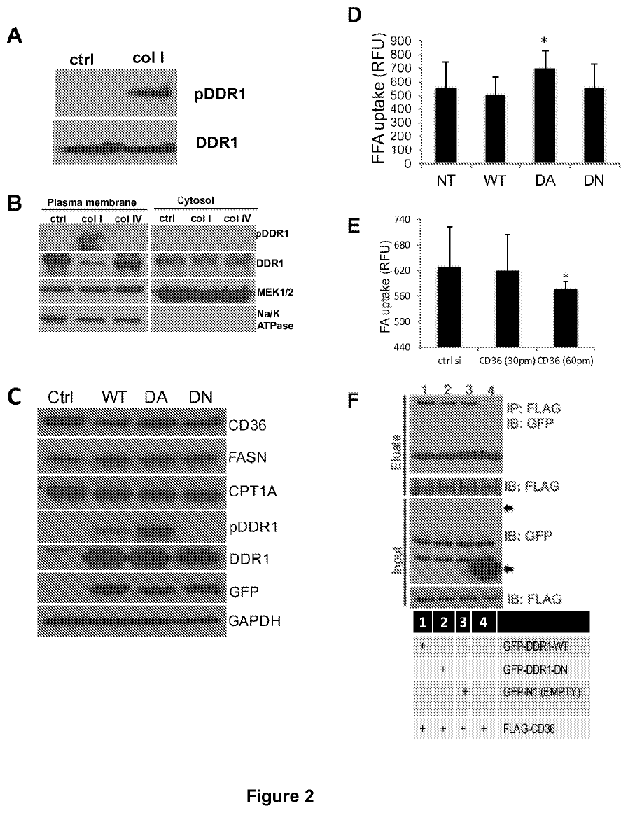

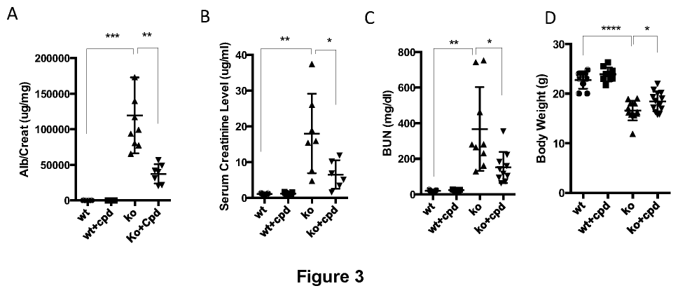

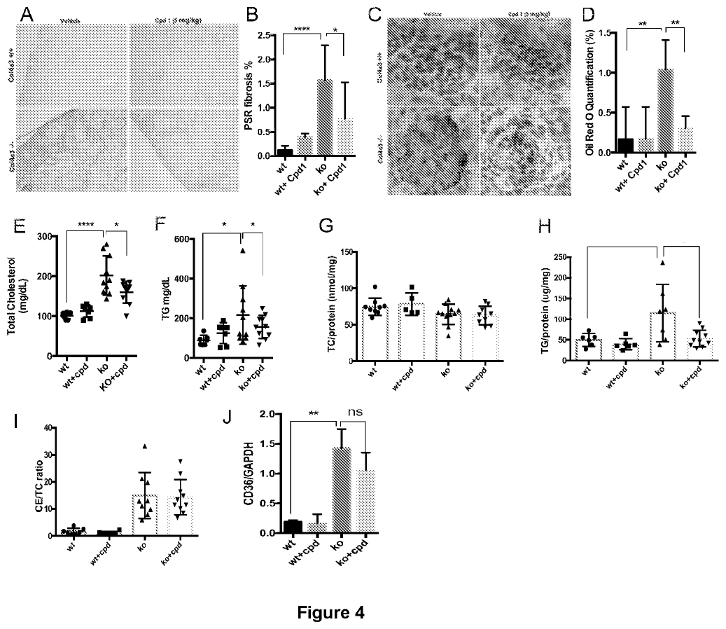

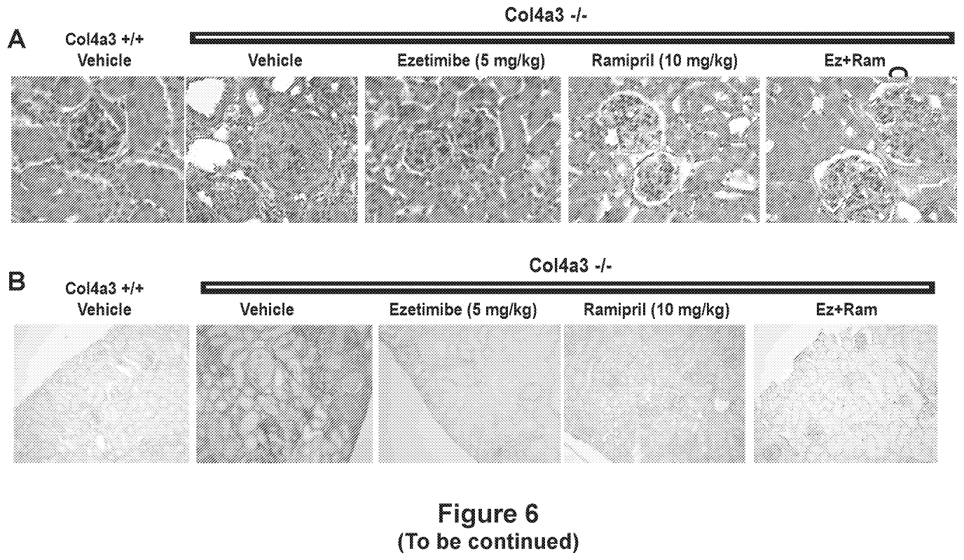

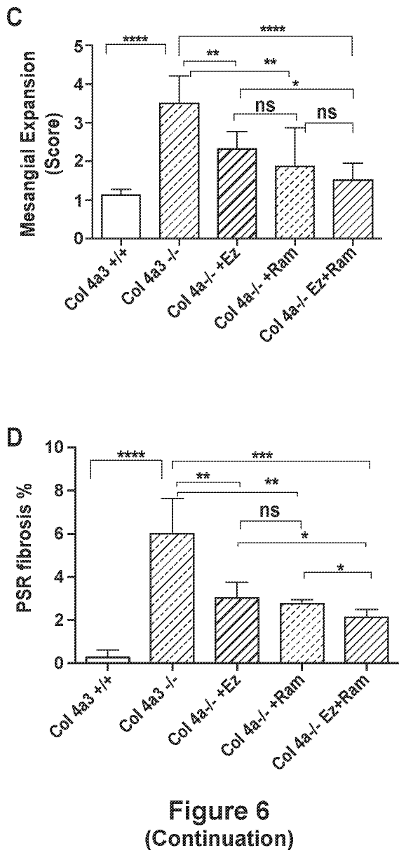

PAS staining was performed to detect and quantify mesangial expansion, Picrossirius Red staining to detect and quantify renal fibrosis in treated compared to untreated Col4a3^{-/-} mice. It was determined that ezetimibe, ramipril and ezetimibe+ramipril significantly reduced mesangial expansion (FIG. 6A,6C) and fibrosis (FIG. 6B,6D) in treated compared to untreated Col4a3^{-/-} mice. Representative images for PAS staining are shown in FIG. 6A and of Picrosirius Red staining to detect fibrosis in FIG. 2B. Bar graph quantifications for both are shown in FIGS. 6C and 6D, respectively. Interestingly, the ezetimibe+ramipril combination further improved renal fibrosis in Col4a3^{-/-}, while no additional effect was observed with regard to an improved mesangial expansion score.

### Example 4—Ramipril Decreased Esterified Cholesterol Content in Renal Cortex of Col4a3 Knockout Mice

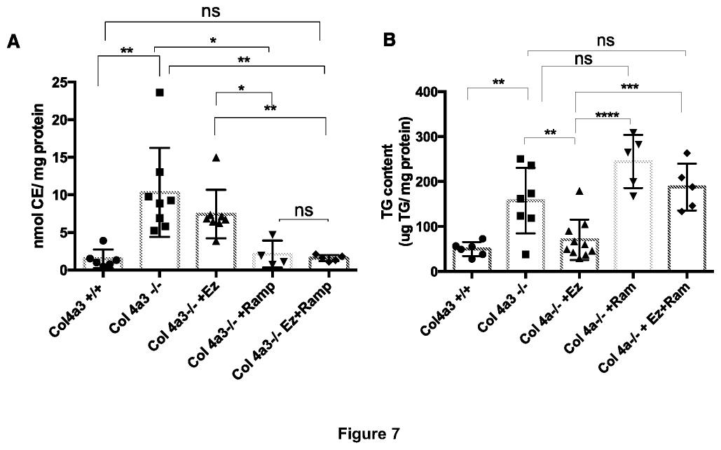

Ramipril is a drug that is used to treat high blood pressure in patients. Ramipril belongs to the class of medications that inhibit angiotensin-converting enzyme (ACE), the enzyme responsible for the conversion of angiotensin I (ATI) to angiotensin II (ATII). ATII regulates blood pressure and is a key component of the renin-angiotensin-aldosterone system (RAAS). It was determined that esterified cholesterol content in kidney cortexes was significantly decreased in ramipril and ramipril+ezetimibe treated Col4a3^{-/-} mice when compared to untreated Col4a3^{-/-} mice (FIG. 7A), which is an unexpected effect as an effect of ramipril on cellular cholesterol metabolism has not yet been described and ramipril is usually considered to have little or no effect, at least on blood cholesterol levels.

As shown in FIG. 5, ezetimibe had no effect on the total cholesterol content. However, it was observed that ezetemibe significantly decreased renal triglyceride content in Col4a3^{-/-} mice when compared to untreated Col4a3^{-/-} mice (FIG. 7B), an effect that was unique as it was not observed when mice were treated with ramipril or with ramipril+ezetimibe.

What is claimed is:

1. A method of treating chronic kidney disease in a subject comprising administering ezetimibe to the subject in an amount effective to treat chronic kidney disease in the subject.

2. The method of claim 1, wherein the ezetimibe reduces triglyceride content in a kidney of the subject.

3. The method of claim 1, wherein the ezetimibe reduces kidney fibrosis in the subject.

4. The method of claim 1, wherein the ezetimibe reduces mesangial expansion in the subject.

5. The method of any one of claims, 1-4, further comprising administering ramipril to the subject.

6. A method of improving renal function in a subject in need thereof comprising administering a combination of ezetimibe and ramipril to the subject in an amount effect to improve renal function.

7. The method of claim 6, wherein the combination reduces esterified cholesterol content in the subject.

8. A method of treating renal disease in a subject suffering from Alport Syndrome comprising administering ezetimibe to the subject in an amount effective to treat the renal disease in the subject.

9. The method of claim 8, further comprising administering ramipril to the subject.

10. The method of any of the preceding claims, wherein the ezetimibe is administered orally.

11. The method of any of the preceding claims, wherein the ramipril is administered orally.

12. A composition comprising ezetimibe, ramipril and a pharmaceutically acceptable carrier or diluent.
